(19) United States
(12) Patent Application Publication
(10) Pub. No.: US 2006/0264511 A1
(43) Pub. Date: Nov. 23, 2006

(54) Method for the treatment of drug-induced sexual dysfunction

(75) Inventor: Robert Pyke, New Fairfield, CT (US)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/383,793

(22) Filed: May 17, 2006

Related U.S. Application Data
(60) Provisional application No. 60/682,760, filed on May 19, 2005.

(51) Int. Cl. A61K 31/192

(52) U.S. Cl. 514/571

(57) ABSTRACT

The invention relates to a method for the treatment of drug-induced sexual dysfunctions comprising the administration of a therapeutically effective amount of flibanserin.

### RELATED APPLICATIONS

This application claims priority to U.S. Provisional Application Ser. No. 60/682,760, filed on May 19, 2005, the contents of which are incorporated by reference in its entirety.

### FIELD OF THE INVENTION

The invention relates to a method for the treatment of drug-induced sexual dysfunction comprising the administration of a therapeutically effective amount of flibanserin.

### DESCRIPTION OF THE INVENTION

Sexual dysfunction is commonly associated with drugs like antihypertensives (V. Pesce, Sexual and Relationship Therapy 17/3: 281-287, 2002 antidepressant drugs (J. R. T. Davidson, Depression 2:233-240 1994,95), antipsychotics (D. Aizenberg, J. clin. Psychiatry 62(7): 541-544, 2001), contraceptives (G. A. Hauser, Geburtshilfe Frauenheilkd. 47 No.12: 859-863, 1987), antineoplastics (G. B. Brock, Drug safety 8 (6): 414-426, 1993), HIV protease inhibitors etc. Patients' complaints relate to sexual dysfunctions like sexual desire disorders, sexual arousal disorders, orgasmic disorders and sexual pain disorders. Several treatments have been tried to alleviate patients' complaints of sexual dysfunction, but only limited success has been reported. Several pharmacological formulations have been used in attempts to intervene in the onset of this unwanted side effect. These attempts at pharmaceutical intervention have not been satisfactory.

One compound class reported to cause sexual dysfunction as a side effect are α-adrenergic receptor antagonists like Prazosin, Clozapine, Mirtazapine, Reboxetine, Clonidine, Guanabenz, Guanethidine, Guanfacine, Guanadrel, Methyldopa, Phenoxibenzamine or Labetalol.

Another compound class reported to cause sexual dysfunction as a side effect are β-adrenergic receptor antagonists like Atenolol, Metoprolol, Nadolol, Timolol, Pindolol or Propranolol.

Another compound class reported to cause sexual dysfunction as a side effect are calcium channel antagonists including Hydralazine, Phenytoin, Nifedipine or Verapamil. Another compound class reported to cause sexual dysfunction as a side effect are sodium channel antagonists including Amiodarone, Carbamazepine or Disopyramide.

A further compound class reported to cause sexual dysfunction as a side effect are 5-HT- and/or norepinephrine (NE)-reuptake inhibitors like Nefazodone, Amfebutamone, Citalopram, Clomipramine, Cericlamine, Femoxetine, Ifoxetine, Fluoxetine, Mianserine, Paroxetine, Cyanodothiepin, Sertraline, Trazodone, Litoxethine, Amitriptyline, Protriptyline, Amoxapine, Desipramine, Dothiepin, Imipramine, Nortriptyline, Venlafaxine, Reserpine or Fluvoxamine.

A further compound class reported to cause sexual dysfunction as a side effect are 5-HT2A antagonists like Ziprasidone or Olanzapine.

A further compound class reported to cause sexual dysfunction as a side effect are D2 antagonists like Droperidol, Metoclopramide, Pimozide, Sulpiride, Quetiapine, Risperidone or Thioridazine.

A further compound class reported to cause sexual dysfunction as a side effect are dopamine agonists like Levodopa, Amineptine, Chlorpromazine, Flupentixol, Fluphenazine, Haloperidol or Perphenazineamitripyline.

Two further compound classes reported to cause sexual dysfunction as a side effect are estrogen and progesterone agonist as used for example in contraceptives like Levonorgestrel, ethinyl estradiol, Desogestrel, Lynoestrenol, Mestranol or Tamoxifene, the latter often used for the treatment of breast cancer.

A further compound class reported to cause sexual dysfunction as a side effect are GABA agonists like Clonazepam, Valproate Flurazepam, Phenobarbital or Prim idone.

A further compound class reported to cause sexual dysfunction as a side effect are HIV protease inhibitors like Indinavir, Nelfinavir, Ritonavir or Saquinavir.

A further compound class reported to cause sexual dysfunction as a side effect are LH-RH modulators (i.e. agonists or antagonists) like Buserelin, Leuprorelin or Danazol.

A further compound class reported to cause sexual dysfunction as a side effect are MAO (Monoamine oxidase) inhibitors like Isocarboxazid, Moclobemide, Phenelzine, or Tranylcypromine.

A further compound class reported to cause sexual dysfunction as a side effect are thiazide diuretics and their analogues like Bendroflumethiazide, Chlortalidone, Hydrochlorothiazide, Trichlormethiazide or Indapamide.

A further compound class reported to cause sexual dysfunction as a side effect are 3-hydroxy-3-methyl-glutaryl-coenzyme A reductase inhibitors like Lovastatin, Rosuvastatin, Fluvastatin, lovastatin, Atorvastatin and Simvastatin.

A further compound class reported to cause sexual dysfunction as a side effect are progestins like Norethindrone, Norgestimate, Norgestrel, Drospirenone and Medroxyprogesterone.

A further compound class reported to cause sexual dysfunction as a side effect are GNRH inhibitors like Abarelix.

A further compound class reported to cause sexual dysfunction as a side effect are GNRH analogues like Triptorelin and Leuprolide.

A further compound class reported to cause sexual dysfunction as a side effect are antiestrogens like Anastrozol, Exemestane, Toremifine, Letrozole and Fulvestrant.

A further compound class reported to cause sexual dysfunction as a side effect are antiandrogens like Bicalutamide and Flutamide.

Other compound classes reported to cause sexual dysfunction as a side effect are potassium channel agonists (e.g. Minoxidil), N-Methyl-D-aspartate (NMDA) receptor antagonists (e.g. Amantadine), muscarinic antagonists (e.g. Atropine, Bethanechol chloride), Na/K ATPase inhibitors (e.g. Digitalis, Digoxin), H/K ATPase inhibitors (proton pump inhibitors) (e.g. Esomeprazole), Histamine H1 or H2 antagonists (e.g. Doxepin, Cimetidine), androgen antagonists (e.g. Cyproterone acetate), aldosterone antagonists (e.g. Spironolactone), calmodulin antagonists (e.g. Trifluoperazine), cholinergic receptor blockers (e.g. Benzatropine), D2 agonists (e.g. Bromocriptine), and other compounds like Reserpine, Triamterene, Stanozolol, Gemfibrozil and Disulfiram.

The compound 1-[2-(4-(3-trifluoromethyl-phenyl)piperazin-1-yl)ethyl]-2,3-dihydro-1H-benzimidazol-2-one (flibanserin) is disclosed in form of its hydrochloride in European Patent Application EP-A-526434 and has the following chemical structure:

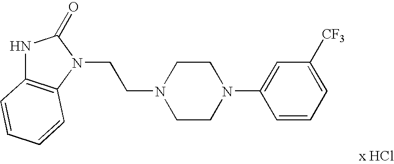

Flibanserin shows affinity for the 5-HT_{1A} and 5-HT₂-receptor. It is therefore a promising therapeutic agent for the treatment of a variety of diseases, for instance depression, schizophrenia and anxiety.

Flibanserin, optionally in form of the free base, the pharmacologically acceptable acid addition salts and/or optionally in form of the hydrates and/or solvates thereof can be used in the treatment of drug-induced sexual dysfunctions.

Therefore, the present invention is directed to a method of treating drug-induced sexual dysfunctions in a patient taking a medication causing sexual dysfunctions comprising administering a therapeutically effective amount of flibanserin, optionally in form of the free base, the pharmacologically acceptable acid addition salts and/or optionally in form of the hydrates and/or solvates thereof to said patient.

As used herein, the term “sexual dysfunction” means a medical diagnosis according to the Diagnostic and Statistical Manual of Mental Disorders, 4th edition, (DSM-IV), Washington DC, American Psychiatric Association, 1996 and includes the criteria, types, disorders, and subtypes of sexual dysfunction listed therein.

The medical diagnosis of sexual dysfunction is clearly described in the Diagnostic and Statistical Manual of Mental Disorders, 4th edition, (DSM-IV), Washington DC, American Psychiatric Association, 1996 (incorporated herein by reference). It includes, sexual desire disorders such as hypoactive sexual desire disorder and sexual aversion disorder; sexual arousal disorders such as female sexual arousal disorder and male erectile disorder; orgasmic disorders such as female orgasmic disorder (formerly, inhibited female orgasm), male orgasmic disorder (formerly, inhibited male orgasm), and premature ejaculation; sexual pain disorders such as drug-induced dyspareunia, drug-induced noncoital sexual pain disorder and drug-induced vaginismus. Drug-induced sexual dysfunction are also included in the DSM-IV.

The term “drug-induced sexual dysfunction” within the present invention refers to a) drug-induced sexual desire disorders like drug-induced female hypoactive sexual desire disorder, drug-induced male hypoactive sexual desire disorder, drug-induced female sexual aversion disorder and drug-induced male sexual aversion disorder, b) drug-induced sexual arousal disorders like drug-induced female sexual arousal disorder and drug-induced male erectile disorder, c) drug-induced orgasmic disorders such as drug-induced female orgasmic disorder (formerly, inhibited female orgasm), drug-induced male orgasmic disorder (formerly, inhibited male orgasm) and drug-induced premature ejaculation as well as d) drug-induced sexual pain disorders like drug-induced dyspareunia, drug-induced noncoital sexual pain disorder and drug-induced vaginismus induced by a medication causing sexual dysfunctions in male or female patients in need of such a medication.

The beneficial effects of flibanserin can be observed regardless of the gender of the patient in need of such treatment.

Accordingly, the instant invention relates to a method for the treatment of drug-induced sexual dysfunctions comprising the administration of a therapeutically effective amount of flibanserin, optionally in form of the free base, the pharmacologically acceptable acid addition salts and/or optionally in form of the hydrates and/or solvates thereof.

In a preferred embodiment the present invention relates to a method for the treatment of drug induced sexual dysfunctions selected from the group consisting of drug-induced sexual desire disorders, drug-induced sexual arousal disorders, drug-induced orgasmic disorders and drug-induced sexual pain disorders.

In a more preferred embodiment the invention relates to a method for the treatment of drug-induced sexual desire disorders selected from the group consisting of drug-induced female hypoactive sexual desire disorder, drug-induced male hypoactive sexual desire disorder, drug-induced female sexual aversion disorder and drug-induced male sexual aversion disorder, comprising the administration of a therapeutically effective amount of flibanserin, optionally in form of the free base, the pharmacologically acceptable acid addition salts and/or optionally in form of the hydrates and/or solvates thereof.

In another preferred embodiment the invention relates to a method for the treatment of drug-induced female hypoactive sexual desire disorder, comprising the administration of a therapeutically effective amount of flibanserin, optionally in form of the free base, the pharmacologically acceptable acid addition salts and/or optionally in form of the hydrates and/or solvates thereof.

In another preferred embodiment the invention relates to a method for the treatment of drug-induced male hypoactive sexual desire disorder, comprising the administration of a therapeutically effective amount of flibanserin, optionally in form of the free base, the pharmacologically acceptable acid addition salts and/or optionally in form of the hydrates and/or solvates thereof.

In another preferred embodiment the invention relates to a method for the treatment of drug-induced female sexual aversion disorder, comprising the administration of a therapeutically effective amount of flibanserin, optionally in form of the free base, the pharmacologically acceptable acid addition salts and/or optionally in form of the hydrates and/or solvates thereof.

In another preferred embodiment the invention relates to a method for the treatment of drug-induced male sexual aversion disorder, comprising the administration of a therapeutically effective amount of flibanserin, optionally in form of the free base, the pharmacologically acceptable acid addition salts and/or optionally in form of the hydrates and/or solvates thereof.

In another preferred embodiment the invention relates to a method for the treatment of drug-induced sexual arousal disorders selected from the group consisting of drug-induced female sexual arousal disorder and drug-induced male erectile disorder, comprising the administration of a therapeutically effective amount of flibanserin, optionally in form of the free base, the pharmacologically acceptable acid addition salts and/or optionally in form of the hydrates and/or solvates thereof.

In another preferred embodiment the invention relates to a method for the treatment of drug-induced female sexual arousal disorder, comprising the administration of a therapeutically effective amount of flibanserin, optionally in form of the free base, the pharmacologically acceptable acid addition salts and/or optionally in form of the hydrates and/or solvates thereof.

In another preferred embodiment the invention relates to a method for the treatment of drug-induced male erectile disorder, comprising the administration of a therapeutically effective amount of flibanserin, optionally in form of the free base, the pharmacologically acceptable acid addition salts and/or optionally in form of the hydrates and/or solvates thereof.

In another preferred embodiment the invention relates to a method for the treatment of drug-induced orgasmic disorders selected from the group consisting of drug-induced female orgasmic disorder, drug-induced male orgasmic disorder and drug-induced premature ejaculation in male, comprising the administration of a therapeutically effective amount of flibanserin, optionally in form of the free base, the pharmacologically acceptable acid addition salts and/or optionally in form of the hydrates and/or solvates thereof.

In another preferred embodiment the invention relates to a method for the treatment of drug-induced female orgasmic disorder, comprising the administration of a therapeutically effective amount of flibanserin, optionally in form of the free base, the pharmacologically acceptable acid addition salts and/or optionally in form of the hydrates and/or solvates thereof.

In another preferred embodiment the invention relates to a method for the treatment of drug-induced male orgasmic disorder, comprising the administration of a therapeutically effective amount of flibanserin, optionally in form of the free base, the pharmacologically acceptable acid addition salts and/or optionally in form of the hydrates and/or solvates thereof.

In another preferred embodiment the invention relates to a method for the treatment of drug-induced premature ejaculation in male, comprising the administration of a therapeutically effective amount of flibanserin, optionally in form of the free base, the pharmacologically acceptable acid addition salts and/or optionally in form of the hydrates and/or solvates thereof.

In another preferred embodiment the invention relates to a method for the treatment of drug-induced sexual pain disorders selected from the group consisting of drug-induced dyspareunia, drug-induced noncoital sexual pain disorder and drug-induced vaginismus, comprising the administration of a therapeutically effective amount of flibanserin, optionally in form of the free base, the pharmacologically acceptable acid addition salts and/or optionally in form of the hydrates and/or solvates thereof.

In another preferred embodiment the invention relates to a method for the treatment of drug-induced dyspareunia, comprising the administration of a therapeutically effective amount of flibanserin, optionally in form of the free base, the pharmacologically acceptable acid addition salts and/or optionally in form of the hydrates and/or solvates thereof.

In another preferred embodiment the invention relates to a method for the treatment of drug-induced noncoital sexual pain disorder, comprising the administration of a therapeutically effective amount of flibanserin, optionally in form of the free base, the pharmacologically acceptable acid addition salts and/or optionally in form of the hydrates and/or solvates thereof.

In another preferred embodiment the invention relates to a method for the treatment of drug-induced vaginismus, comprising the administration of a therapeutically effective amount of flibanserin, optionally in form of the free base, the pharmacologically acceptable acid addition salts and/or optionally in form of the hydrates and/or solvates thereof.

In a particular preferred embodiment the invention relates to a method for the treatment of drug-induced female hypoactive sexual desire disorder, comprising the administration of a therapeutically effective amount of flibanserin, optionally in form of the free base, the pharmacologically acceptable acid addition salts and/or optionally in form of the hydrates and/or solvates thereof.

Another embodiment of the present invention relates to the use of flibanserin, optionally in form of the free base, the pharmacologically acceptable acid addition salts and/or optionally in form of the hydrates and/or solvates thereof, for the preparation of a medicament for the treatment of the aforementioned drug-induced dysfunctions.

In another preferred embodiment the invention relates to a method for the treatment of any of the aforementioned drug-induced dysfunctions wherein the dysfunction has been induced by a compound selected from the group consisting of α-adrenergic receptor antagonists, β-adrenergic receptor antagonists, calcium channel antagonists, sodium channel antagonists, 5-HT- and/or norepinephrine (NE)—reuptake inhibitors, 5-HT2A antagonists, D2 antagonists, dopamine agonists, Estrogen agonists, progesterone agonists, GABA agonists, HIV protease inhibitors, LH-RH modulators, MAO inhibitors, thiazide diuretics, potassium channel agonists, N-Methyl-D-aspartate (NMDA) receptor antagonists, muscarinic antagonists, Na/K-ATPase inhibitors, H/K-ATPase inhibitors, Histamine H1 or H2 antagonists, androgen antagonists, aldosterone antagonists, calmodulin antagonists, cholinergic receptor blockers, D2 agonists, 3-hydroxy-3-methyl-glutaryl-coenzyme A reductase inhibitors, progestins, GNRH inhibitors, GNRH analogues, antiestrogens and antiandrogens.

In a preferred embodiment the invention relates to a method for the treatment of the aforementioned drug-induced dysfunctions wherein the dysfunction has been induced by α-adrenergic receptor antagonists, more preferably by Prazosin, Clozapine, Mirtazapine, Reboxetine, Clonidine, Guanabenz, Guanethidine, Guanfacine, Guanadrel, Methyldopa, Phenoxibenzamine or Labetalol.

In another preferred embodiment the invention relates to a method for the treatment of the aforementioned drug-induced dysfunctions wherein the dysfunction has been induced by β-adrenergic receptor antagonists, more preferably by Atenolol, Metoprolol, Nadolol, Timolol, Pindolol or Propranolol.

In another preferred embodiment the invention relates to a method for the treatment of the aforementioned drug-induced dysfunctions wherein the dysfunction has been induced by calcium channel antagonists, more preferably by Hydralazine, Phenytoin, Nifedipine or Verapamil.

In another preferred embodiment the invention relates to a method for the treatment of the aforementioned drug-induced dysfunctions wherein the dysfunction has been induced by sodium channel antagonists, more preferably by Amiodarone, Carbamazepine or Disopyramide.

In another preferred embodiment the invention relates to a method for the treatment of the aforementioned drug-induced dysfunctions wherein the dysfunction has been induced by 5-HT- and/or norepinephrine (NE)-reuptake inhibitors, more preferably by Nefazodone, Amfebutamone, Citalopram, Clomipramine, Fluoxetine, Mianserine, Paroxetine, Sertraline, Trazodone, Amitriptyline, Protriptyline, Amoxapine, Desipramine, Dothiepin, Imipramine, Nortriptyline, Venlafaxine, Reserpine or Fluvoxamine.

In another preferred embodiment the invention relates to a method for the treatment of the aforementioned drug-induced dysfunctions wherein the dysfunction has been induced by 5-HT_{2A} antagonists, more preferably by Ziprasidone or Olanzapine.

In another preferred embodiment the invention relates to a method for the treatment of the aforementioned drug-induced dysfunctions wherein the dysfunction has been induced by D2 antagonists, more preferably by Droperidol, Metoclopramide, Pimozide, Sulpiride, Quetiapine, Risperidone or Thioridazine.

In another preferred embodiment the invention relates to a method for the treatment of the aforementioned drug-induced dysfunctions wherein the dysfunction has been induced by dopamine agonists, more preferably by Levodopa, Amineptine, Chlorpromazine, Flupentixol, Fluphenazine, Haloperidol or Perphenazineamitripyline.

In another preferred embodiment the invention relates to a method for the treatment of the aforementioned drug-induced dysfunctions wherein the dysfunction has been induced by estrogen and/or progesterone agonists, more preferably by Levonorgestrel, ethinyl estradiol, Desogestrel, Lynoestrenol, Mestranol or Tamoxifene.

In another preferred embodiment the invention relates to a method for the treatment of the aforementioned drug-induced dysfunctions wherein the dysfunction has been induced by GABA agonists, more preferably by Clonazepam, Valproate Flurazepam, Phenobarbital or Primidone.

In another preferred embodiment the invention relates to a method for the treatment of the aforementioned drug-induced dysfunctions wherein the dysfunction has been induced by HIV protease inhibitors, more preferably by Indinavir, Nelfinavir, Ritonavir or Saquinavir.

In another preferred embodiment the invention relates to a method for the treatment of the aforementioned drug-induced dysfunctions wherein the dysfunction has been induced by LH-RH modulators, more preferably by Buserelin, Leuprorelin or Danazol.

In another preferred embodiment the invention relates to a method for the treatment of the aforementioned drug-induced dysfunctions wherein the dysfunction has been induced by MAO-inhibitors, more preferably by Isocarboxazid, Moclobemide, Phenelzine, or Tranylcypromine.

In another preferred embodiment the invention relates to a method for the treatment of the aforementioned drug-induced dysfunctions wherein the dysfunction has been induced by thiazide diuretics and their analogues, more preferably by Bendroflumethiazide, Chlortalidone, Hydrochlorothiazide, Trichlormethiazide or Indapamide.

In another preferred embodiment the invention relates to a method for the treatment of the aforementioned drug-induced dysfunctions wherein the dysfunction has been induced by potassium channel agonists, more preferably by Minoxidil.

In another preferred embodiment the invention relates to a method for the treatment of the aforementioned drug-induced dysfunctions wherein the dysfunction has been induced by N-Methyl-D-aspartate (NMDA) receptor antagonists, more preferably by Amantadine.

In another preferred embodiment the invention relates to a method for the treatment of the aforementioned drug-induced dysfunctions wherein the dysfunction has been induced by muscarinic antagonists, more preferably by Atropine or Bethanechol chloride.

In another preferred embodiment the invention relates to a method for the treatment of the aforementioned drug-induced dysfunctions wherein the dysfunction has been induced by Na/K ATPase inhibitors, more preferably by Digitalis or Digoxin.

In another preferred embodiment the invention relates to a method for the treatment of the aforementioned drug-induced dysfunctions wherein the dysfunction has been induced by H/K ATPase inhibitors, more preferably by Esomeprazole.

In another preferred embodiment the invention relates to a method for the treatment of the aforementioned drug-induced dysfunctions wherein the dysfunction has been induced by Histamine H1 or H2 antagonists, more preferably by Doxepin or Cimetidine.

In another preferred embodiment the invention relates to a method for the treatment of the aforementioned drug-induced dysfunctions wherein the dysfunction has been induced by androgen antagonists, more preferably by Cyproterone acetate.

In another preferred embodiment the invention relates to a method for the treatment of the aforementioned drug-induced dysfunctions wherein the dysfunction has been induced by aldosterone antagonists, more preferably by Spironolactone.

In another preferred embodiment the invention relates to a method for the treatment of the aforementioned drug-induced dysfunctions wherein the dysfunction has been induced by calmodulin antagonists, more preferably by Trifluoperazine.

In another preferred embodiment the invention relates to a method for the treatment of the aforementioned drug-induced dysfunctions wherein the dysfunction has been induced by cholinergic receptor blockers, more preferably by Benzatropine.

In another preferred embodiment the invention relates to a method for the treatment of the aforementioned drug-induced dysfunctions wherein the dysfunction has been induced by D2 agonists, more preferably by Bromocriptine.

In another preferred embodiment the invention relates to a method for the treatment of the aforementioned drug-induced dysfunctions wherein the dysfunction has been induced by 3-hydroxy-3-methyl-glutaryl-coenzyme A reductase inhibitors, more preferably by Lovastatin, Rosuvastatin, Fluvastatin, lovastatin, Atorvastatin or Simvastatin.

In another preferred embodiment the invention relates to a method for the treatment of the aforementioned drug-induced dysfunctions wherein the dysfunction has been induced by progestins, more preferably by Norethindrone, Norgestimate, Norgestrel, Drospirenone or Medroxyprogesterone.

In another preferred embodiment the invention relates to a method for the treatment of the aforementioned drug-induced dysfunctions wherein the dysfunction has been induced by GNRH inhibitors, more preferably by Abarelix.

In another preferred embodiment the invention relates to a method for the treatment of the aforementioned drug-induced dysfunctions wherein the dysfunction has been induced by GNRH analogues, more preferably by Triptorelin or Leuprolide.

In another preferred embodiment the invention relates to a method for the treatment of the aforementioned drug-induced dysfunctions wherein the dysfunction has been induced by antiestrogens, more preferably by Anastrozol, Exemestane, Toremifine, Letrozole or Fulvestrant.

In another preferred embodiment the invention relates to a method for the treatment of the aforementioned drug-induced dysfunctions wherein the dysfunction has been induced by antiandrogens, more preferably by Bicalutamide or Flutamide.

In another preferred embodiment the invention relates to a method for the treatment of the aforementioned drug-induced dysfunctions wherein the dysfunction has been induced by a compound selected from the group consisting of Reserpine, Triamterene, Stanozolol, Gemfibrozil and Disulfiram.

Therefore, in a more preferred embodiment the present invention relates to to a method for the treatment of the aforementioned drug-induced dysfunctions wherein the dysfunction has been induced by a compound selected from the group consisting of Prazosin, Clozapine, Mirtazapine, Reboxetine, Clonidine, Guanabenz, Guanethidine, Guanfacine, Guanadrel, Methyldopa, Phenoxibenzamine, Labetalol, Atenolol, Metoprolol, Nadolol, Timolol, Pindolol, Propranolol, Hydralazine, Phenytoin, Nifedipine, Verapamil, Amiodarone, Carbamazepine, Disopyramide, Nefazodone, Amfebutamone, Citalopram, Clomipramine, Fluoxetine, Mianserine, Paroxetine, Sertraline, Trazodone, Amitriptyline, Protriptyline, Amoxapine, Desipramine, Dothiepin, Imipramine, Nortriptyline, Venlafaxine, Reserpine, Fluvoxamine, Ziprasidone, Olanzapine, Droperidol, Metoclopramide, Pimozide, Sulpiride, Quetiapine, Risperidone, Thioridazine, Levodopa, Amineptine, Chlorpromazine, Flupentixol, Fluphenazine, Haloperidol, Perphenazineamitripyline, Levonorgestrel, ethinyl estradiol, Desogestrel, Lynoestrenol, Mestranol, Tamoxifene, Clonazepam, Valproate, Flurazepam, Phenobarbital, Primidone, Indinavir, Nelfinavir, Ritonavir, Saquinavir, Isocarboxazid, Moclobemide, Pheneizine, Tranylcypromine, Bendroflumethiazide, Chlortalidone, Hydrochlorothiazide, Trichlormethiazide, Indapamide, Minoxidil, Amantadine, Atropine, Bethanechol chloride, Digitalis, Digoxin, Esomeprazole, Doxepin, Cimetidine, Cyproterone acetate, Spironolactone, Trifluoperazine, Benzatropine, Bromocriptine, Reserpine, Triamterene, Stanozolol, Gemfibrozil, Disulfiram, Lovastatin, Rosuvastatin, Fluvastatin, lovastatin, Atorvastatin, Simvastatin, Norethindrone, Norgestimate, Norgestrel, Drospirenone, Medroxyprogesterone, Abarelix, Triptorelin, Leuprolide, Anastrozol, Exemestane, Toremifine, Letrozole, Fulvestrant, Bicalutamide or Flutamide.

As the present invention provides methods for the treatment of drug induced sexual dysfunctions, the invention also relates to combining separate pharmaceutical compositions in kit form. Therefore, in a further embodiment the present invention provides a kit comprising a) a first pharmaceutical composition comprising an active ingredient, which has the side effect of causing sexual dysfunctions, for the treatment of an underlying disease; b) a second pharmaceutical composition comprising flibanserin, optionally in form of the free base, the pharmacologically acceptable acid addition salts and/or optionally in form of the hydrates and/or solvates thereof for the treatment of the sexual dysfunctions induced by the active ingredient of the first pharmaceutical composition; and a container for both compositions.

In a further embodiment the present invention provides a kit comprising a) a first pharmaceutical composition comprising an active ingredient, which has the side effect of causing sexual dysfunctions, for the treatment of an underlying disease, wherein the active ingredient is selected from the group consisting of α-adrenergic receptor antagonists, β-adrenergic receptor antagonists, calcium channel antagonists, sodium channel antagonists, 5-HT- and/or norepinephrine (NE)—reuptake inhibitors, 5-HT2A antagonists, D2 antagonists, dopamine agonists, Estrogen agonists, progesterone agonists, GABA agonists, HIV protease inhibitors, LH-RH modulators, MAO inhibitors, thiazide diuretics, potassium channel agonists, N-Methyl-D-aspartate (NMDA) receptor antagonists, muscarinic antagonists, Na/K-ATPase inhibitors, H/K-ATPase inhibitors, Histamine H1 or H2 antagonists, androgen antagonists, aldosterone antagonists, calmodulin antagonists, cholinergic receptor blockers, D2 agonists, 3-hydroxy-3-methyl-glutaryl-coenzyme A reductase inhibitors, progestins, GNRH inhibitors, GNRH analogues, antiestrogens and antiandrogens; b) a second pharmaceutical composition comprising flibanserin, optionally in form of the free base, the pharmacologically acceptable acid addition salts and/or optionally in form of the hydrates and/or solvates thereof for the treatment of the sexual dysfunctions induced by the active ingredient of the first pharmaceutical composition; and a container for both compositions.

In a further embodiment the present invention provides a kit comprising a) a first pharmaceutical composition comprising an active ingredient, which has the side effect of causing sexual dysfunctions, for the treatment of an underlying disease, wherein the active ingredient is selected from the group consisting of Prazosin, Clozapine, Mirtazapine, Reboxetine, Clonidine, Guanabenz, Guanethidine, Guanfacine, Guanadrel, Methyldopa, Phenoxibenzamine, Labetalol, Atenolol, Metoprolol, Nadolol, Timolol, Pindolol, Propranolol, Hydralazine, Phenytoin, Nifedipine, Verapamil, Amiodarone, Carbamazepine, Disopyramide, Nefazodone, Amfebutamone, Citalopram, Clomipramine, Fluoxetine, Mianserine, Paroxetine, Sertraline, Trazodone, Amitriptyline, Protriptyline, Amoxapine, Desipramine, Dothiepin, Imipramine, Nortriptyline, Venlafaxine, Reserpine, Fluvoxamine, Ziprasidone, Olanzapine, Droperidol, Metoclopramide, Pimozide, Sulpiride, Quetiapine, Risperidone, Thioridazine, Levodopa, Amineptine, Chlorpromazine, Flupentixol, Fluphenazine, Haloperidol, Perphenazineamitripyline, Levonorgestrel, ethinyl estradiol, Desogestrel, Lynoestrenol, Mestranol, Tamoxifene, Clonazepam, Valproate, Flurazepam, Phenobarbital, Primidone, Indinavir, Nelfinavir, Ritonavir, Saquinavir, Isocarboxazid, Moclobemide, Pheneizine, Tranylcypromine, Bendroflumethiazide, Chlortalidone, Hydrochlorothiazide, Trichlormethiazide, Indapamide, Minoxidil, Amantadine, Atropine, Bethanechol chloride, Digitalis, Digoxin, Esomeprazole, Doxepin, Cimetidine, Cyproterone acetate, Spironolactone, Trifluoperazine, Benzatropine, Bromocriptine, Reserpine, Triamterene, Stanozolol, Gemfibrozil, Disulfiram, Lovastatin, Rosuvastatin, Fluvastatin, lovastatin, Atorvastatin, Simvastatin, Norethindrone, Norgestimate, Norgestrel, Drospirenone, Medroxyprogesterone, Abarelix, Triptorelin, Leuprolide, Anastrozol, Exemestane, Toremifine, Letrozole, Fulvestrant, Bicalutamide or Flutamide; b) a second pharmaceutical composition comprising flibanserin, optionally in form of the free base, the pharmacologically acceptable acid addition salts and/or optionally in form of the hydrates and/or solvates thereof for the treatment of the sexual dysfunctions induced by the active ingredient of the first pharmaceutical composition; and a container for both compositions.

The term “co-administration”, within the present invention means that both active ingredients mentioned above can be taken from the kit and combined for administration together as a composition or as part of the same, unitary dosage form, such as an parenterally or orally administered solution. “Co-administration” also includes administering the components separately (e.g. as tablets or capsules), but as part of the same therapeutic treatment program or regimen. Both components need not be administered at essentially the same time, although they can be if so desired. Thus “co-administration” includes, for example administering all active ingredients as separate dosages or dosage forms and at essentially the same time. The term also includes separate administration at different times, in any order, and if preferred by different routes of administration. An example of a kit is the so-called blister pack well known in the packaging industry particularly for packaging pharmaceutical dosage forms.

Instead of a kit, the active ingredients causing sexual dysfunctions as a side effect and flibanserin can be combined in one dosage form. Therefore, the present invention also relates to compositions comprising an active ingredient causing sexual dysfunctions as a side effect and flibanserin, optionally in form of the free base, the pharmacologically acceptable acid addition salts and/or optionally in form of the hydrates and/or solvates thereof.

Accordingly, the present invention also relates to compositions comprising a) an active ingredient causing sexual dysfunctions as a side effect and b) flibanserin, optionally in form of the free base, the pharmacologically acceptable acid addition salts and/or optionally in form of the hydrates and/or solvates thereof.

Accordingly, the present invention also relates to compositions comprising a) an active ingredient causing sexual dysfunctions as a side effect, wherein the active ingredient is selected from the group consisting of α-adrenergic receptor antagonists, β-adrenergic receptor antagonists, calcium channel antagonists, sodium channel antagonists, 5-HT- and/or norepinephrine (NE)-reuptake inhibitors, 5-HT2A antagonists, D2 antagonists, dopamine agonists, Estrogen agonists, progesterone agonists, GABA agonists, HIV protease inhibitors, LH-RH modulators, MAO inhibitors, thiazide diuretics, potassium channel agonists, N-Methyl-D-aspartate (NMDA) receptor antagonists, muscarinic antagonists, Na/K-ATPase inhibitors, H/K-ATPase inhibitors, Histamine H1 or H2 antagonists, androgen antagonists, aldosterone antagonists, calmodulin antagonists, cholinergic receptor blockers, D2 agonists, 3-hydroxy-3-methyl-glutaryl-coenzyme A reductase inhibitors, progestins, GNRH inhibitors, GNRH analogues, antiestrogens and antiandrogens and b) flibanserin, optionally in form of the free base, the pharmacologically acceptable acid addition salts and/or optionally in form of the hydrates and/or solvates thereof.

The present invention also relates to compositions comprising a) an active ingredient causing sexual dysfunctions as a side effect, wherein the active ingredient is selected from the group consisting of Prazosin, Clozapine, Mirtazapine, Reboxetine, Clonidine, Guanabenz, Guanethidine, Guanfacine, Guanadrel, Methyldopa, Phenoxibenzamine, Labetalol, Atenolol, Metoprolol, Nadolol, Timolol, Pindolol, Propranolol, Hydralazine, Phenytoin, Nifedipine, Verapamil, Amiodarone, Carbamazepine, Disopyramide, Nefazodone, Amfebutamone, Citalopram, Clomipramine, Fluoxetine, Mianserine, Paroxetine, Sertraline, Trazodone, Amitriptyline, Protriptyline, Amoxapine, Desipramine, Dothiepin, Imipramine, Nortriptyline, Venlafaxine, Reserpine, Fluvoxamine, Ziprasidone, Olanzapine, Droperidol, Metoclopramide, Pimozide, Sulpiride, Quetiapine, Risperidone, Thioridazine, Levodopa, Amineptine, Chlorpromazine, Flupentixol, Fluphenazine, Haloperidol, Perphenazineamitripyline, Levonorgestrel, ethinyl estradiol, Desogestrel, Lynoestrenol, Mestranol, Tamoxifene, Clonazepam, Valproate, Flurazepam, Phenobarbital, Primidone, Indinavir, Nelfinavir, Ritonavir, Saquinavir, Isocarboxazid, Moclobemide, Pheneizine, Tranylcypromine, Bendroflumethiazide, Chlortalidone, Hydrochlorothiazide, Trichlormethiazide, Indapamide, Minoxidil, Amantadine, Atropine, Bethanechol chloride, Digitalis, Digoxin, Esomeprazole, Doxepin, Cimetidine, Cyproterone acetate, Spironolactone, Trifluoperazine, Benzatropine, Bromocriptine, Reserpine, Triamterene, Stanozolol, Gemfibrozil, Disulfiram, Lovastatin, Rosuvastatin, Fluvastatin, lovastatin, Atorvastatin, Simvastatin, Norethindrone, Norgestimate, Norgestrel, Drospirenone, Medroxyprogesterone, Abarelix, Triptorelin, Leuprolide, Anastrozol, Exemestane, Toremifine, Letrozole, Fulvestrant, Bicalutamide or Flutamide and b) flibanserin, optionally in form of the free base, the pharmacologically acceptable acid addition salts and/or optionally in form of the hydrates and/or solvates thereof.

As already mentioned above, flibanserin may be used in form of the free base, optionally in form of its pharmaceutically acceptable acid addition salts and/or optionally in form of the hydrates and/or solvates thereof. Suitable acid addition salts include for example those of the acids selected from, succinic acid, hydrobromic acid, acetic acid, fumaric acid, maleic acid, methanesulphonic acid, lactic acid, phosphoric acid, hydrochloric acid, sulphuric acid, tartaric acid and citric acid. Mixtures of the abovementioned acid addition salts may also be used. From the aforementioned acid addition salts the hydrochloride and the hydrobromide, particularly the hydrochloride, are preferred. If flibanserin is used in form of the free base, it is preferably used in form of flibanserin polymorph A as disclosed in WO 03/014079.

The active ingredients causing sexual dysfunctions which are suitable to be combined with flibanserin within the teaching of the instant invention and which are mentioned hereinbefore may also be capable of forming acid addition salts with pharmaceutically acceptable acids. Representative salts include the following: Acetate, Benzenesulfonate, Benzoate, Bicarbonate, Bisulfate, Bitartrate, Borate, Bromide, Camsylate, Carbonate, Chloride, Clavulanate, Citrate, Dihydrochloride, Edetate, Edisylate, Estolate, Esylate, Fumarate, Gluceptate, Gluconate,Glutamate, Glycollylarsanilate, Hexylresorcinate, Hydrabamine, Hydrobromide, Hydrochloride, Hydroxynaphthoate, Iodide, Isothionate, Lactate, Lactobionate, Laurate, Malate, Maleate, Mandelate, Mesylate, Methylbromide, Methylnitrate, Methylsulfate, Mucate,Napsylate, Nitrate, N-methylglucamine ammonium salt, Oleate, Oxalate, Pamoate (Embonate), Palmitate, Pantothenate, Phosphate/diphosphate, Polygalacturonate, Salicylate, Stearate, Sulfate, Subacetate, Succinate, Tannate, Tartrate, Teoclate, Tosylate, Triethiodide and Valerate.

Furthermore, where the compounds causing sexual dysfunctions carry an acidic moiety, suitable pharmaceutically acceptable salts thereof may include alkali metal salts, e.g., sodium or potassium salts; alkaline earth metal salts, e. g., calcium or magnesium salts; and salts formed with suitable organic ligands, e.g., quaternary ammonium salts.

The compounds causing sexual dysfunctions may have chiral centers and occur as racemates, racemic mixtures and as individual diastereomers, or enantiomers with all isomeric forms being included in the present invention. Therefore, where a compound is chiral, the separate enantiomers, substantially free of the other, are included within the scope of the invention. Further included are all mixtures of the two enantiomers. Also included within the scope of the invention are polymorphs and hydrates of the compounds of the instant invention.

The present invention includes within its scope prodrugs of flibanserin and the compounds causing sexual dysfunctions. In general, such prodrugs will be functional derivatives of the compounds of this invention which are readily convertible in vivo into the required compound.

Flibanserin, optionally used in form of its pharmaceutically acceptable acid addition salts and/or optionally in form of the hydrates and/or solvates thereof, or in form of flibanserin polymorph A, as well as the active ingredients causing sexual dysfunction as a side effect may be incorporated into the conventional pharmaceutical preparation in solid, liquid or spray form. The compositions may, for example, be presented in a form suitable for oral, rectal, parenteral administration or for nasal inhalation: preferred forms includes for example, capsules, tablets, coated tablets, ampoules, suppositories and nasal spray.

The active ingredients may be incorporated in excipients or carriers conventionally used in pharmaceutical compositions such as, for example, talc, arabic gum, lactose, gelatine, magnesium stearate, corn starch, acqueous or non acqueous vehicles, polyvynil pyrrolidone, semisynthetic glicerides of fatty acids, benzalconium chloride, sodium phosphate, EDTA, polysorbate 80. The compositions are advantageously formulated in dosage units, each dosage unit being adapted to supply a single dose of the active ingredient. The dosis range of flibanserin applicable per day is between 0.1 to 400, preferably between 1.0 to 300, more preferably between 2 to 200 mg. Each dosage unit may conveniently contain from 0,01 mg to 100 mg, preferably from 0,1 to 50 mg.

The concentration of active compounds causing sexual dysfunction in the composition will depend on absorption, inactivation, and excretion rates of the active compound, the dosage schedule, and amount administered as well as other factors known to those of skill in the art. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number or layers to achieve delayed release, possibly using the excipients mentioned above for the tablets.

Syrups or elixirs containing the active substances or combinations thereof according to the invention may additionally contain a sweetener such as saccharine, cyclamate, glycerol or sugar and a flavour enhancer, e.g of. a flavouring such as vanilline or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions for injection are prepared in the usual way, e.g of. with the addition of preservatives such as p-hydroxybenzoates, or stabilisers such as alkali metal salts of ethylenediamine tetraacetic acid, and transferred into injection vials or ampoules.

Capsules containing one or more active substances or combinations of active substances may for example be prepared by mixing the active substances with inert carriers such as lactose or sorbitol and packing them into gelatine capsules.

Suitable suppositories may be made for example by mixing with carriers provided for this purpose, such as neutral fats or polyethyleneglycol or the derivatives thereof.

The Examples which follow illustrate the present invention without restricting its scope:

Examples of Pharmaceutical Formulations

A) Tablets

| | per tablet |
|---|---|
| flibanserin hydrochloride | 100 mg |
| lactose | 240 mg |
| corn starch | 340 mg |
| polyvinylpyrrolidone | 45 mg |
| magnesium stearate | 15 mg |
| | 740 mg |

The finely ground active substance, lactose and some of the corn starch are mixed together. The mixture is screened, then moistened with a solution of polyvinylpyrrolidone in water, kneaded, wet-granulated and dried. The granules, the remaining corn starch and the magnesium stearate are screened and mixed together. The mixture is compressed to produce tablets of suitable shape and size.

B) Tablets

| | per tablet |
|---|---|
| flibanserin hydrochloride | 80 mg |
| corn starch | 190 mg |
| lactose | 55 mg |
| microcrystalline cellulose | 35 mg |
| polyvinylpyrrolidone | 15 mg |
| sodium-carboxymethyl starch | 23 mg |
| magnesium stearate | 2 mg |
| | 400 mg |

The finely ground active substance, some of the corn starch, lactose, microcrystalline cellulose and polyvinylpyrrolidone are mixed together, the mixture is screened and worked with the remaining corn starch and water to form a granulate which is dried and screened. The sodium-carboxymethyl starch and the magnesium stearate are added and mixed in and the mixture is compressed to form tablets of a suitable size.

C) Coated tablets

| | per coated tablet |
|---|---|
| flibanserin hydrochloride | 5 mg |
| corn starch | 41.5 mg |
| lactose | 30 mg |
| polyvinylpyrrolidone | 3 mg |
| magnesium stearate | 0.5 mg |
| | 80 mg |

The active substance, corn starch, lactose and polyvinylpyrrolidone are thoroughly mixed and moistened with water. The moist mass is pushed through a screen with a 1 mm mesh size, dried at about 45° C and the granules are then passed through the same screen. After the magnesium stearate has been mixed in, convex tablet cores with a diameter of 6 mm are compressed in a tablet-making machine . The tablet cores thus produced are coated in known manner with a covering consisting essentially of sugar and talc. The finished coated tablets are polished with wax.

D) Capsules

| | per capsule |
|---|---|
| flibanserin hydrochloride | 150 mg |
| Corn starch | 268.5 mg |
| Magnesium stearate | 1.5 mg |
| | 420 mg |

The substance and corn starch are mixed and moistened with water. The moist mass is screened and dried. The dry granules are screened and mixed with magnesium stearate. The finished mixture is packed into size 1 hard gelatine capsules.

E) Ampoule solution

| | |
|---|---|
| flibanserin hydrochloride | 50 mg |
| sodium chloride | 50 mg |
| water for inj. | 5 ml |

The active substance is dissolved in water at its own pH or optionally at pH 5.5 to 6.5 and sodium chloride is added to make it isotonic. The solution obtained is filtered free from pyrogens and the filtrate is transferred under aseptic conditions into ampoules which are then sterilised and sealed by fusion.

F) Suppositories

| | |
|---|---|
| flibanserin hydrochloride | 50 mg |
| solid fat | 1650 mg |
| | 1700 mg |

The hard fat is melted. At 40° C. the ground active substance is homogeneously dispersed. It is cooled to 38° C. and poured into slightly chilled suppository moulds.

In a particular preferred embodiment of the instant invention, flibanserin is administered in form of specific film coated tablets. Examples of these preferred formulations are listed below. The film coated tablets listed below can be manufactured according to procedures known in the art (see hereto WO 03/097058).

G) Film coated tablet

| Constituents | mg/tablet |
|---|---|
| Core | |
| Flibanserin | 25.000 |
| Lactose monohydrate | 71.720 |
| Microcrystalline cellulose | 23.905 |
| HPMC (Methocel E5) | 1.250 |
| Carboxymethylcellulose sodium | 2.500 |
| Magnesium stearate | 0.625 |
| Coating | |
| HPMC (Methocel E5) | 1.440 |
| Polyethylene Glycol 6000 | 0.420 |
| Titanium dioxide | 0.600 |
| Talc | 0.514 |
| Iron oxide red | 0.026 |
| Total Film coated tablet | 128.000 |

H) Film coated tablet

| Constituents | mg/tablet |
|---|---|
| Core | |
| Flibanserin | 50.000 |
| Lactose monohydrate | 143.440 |
| Microcrystalline cellulose | 47.810 |
| HPMC (e.g. Pharmacoat 606) | 2.500 |
| Carboxymethylcellulose sodium | 5.000 |
| Magnesium stearate | 1.250 |
| Coating | |
| HPMC (e.g. Pharmacoat 606) | 2.400 |
| Polyethylene Glycol 6000 | 0.700 |
| Titanium dioxide | 1.000 |
| Talc | 0.857 |
| Iron oxide red | 0.043 |
| Total Film coated tablet | 255.000 |

I) Film coated tablet

| Constituents | mg/tablet |
|---|---|
| Core | |
| Flibanserin | 100.000 |
| Lactose monohydrate | 171.080 |
| Microcrystalline cellulose | 57.020 |
| HPMC (e.g. Methocel E5) | 3.400 |
| Carboxymethylcellulose sodium | 6.800 |
| Magnesium stearate | 1.700 |
| Coating | |
| HPMC (e.g. Methocel E5) | 3.360 |
| Polyethylene Glycol 6000 | 0.980 |
| Titanium dioxide | 1.400 |
| Talc | 1.200 |
| Iron oxide red | 0.060 |
| Total Film coated tablet | 347.000 |

J) Film coated tablet

| Constituents | mg/tablet |
|---|---|
| Core | |
| Flibanserin | 2.000 |
| Dibasic Calciumphosphate, anhydrous | 61.010 |
| Microcrystalline cellulose | 61.010 |
| HPMC (Methocel E5) | 1.950 |
| Carboxymethylcellulose sodium | 2.600 |
| Colloidal silicon dioxide | 0.650 |
| Magnesium stearate | 0.780 |
| Coating | |
| HPMC (Methocel E5) | 1.440 |
| Polyethylene Glycol 6000 | 0.420 |
| Titanium dioxide | 0.600 |
| Talc | 0.514 |
| Iron oxide red | 0.026 |
| Total Film coated tablet | 133.000 |

K) Film coated tablet

| Constituents | mg/tablet |
|---|---|
| Core | |
| Flibanserin | 100.000 |
| Dibasic Calciumphosphate, anhydrous | 69.750 |
| Microcrystalline cellulose | 69.750 |
| HPMC (e.g. Methocel E5) | 2.750 |
| Carboxymethylcellulose sodium | 5.000 |
| Colloidal silicon dioxide | 1.250 |
| Magnesium stearate | 1.500 |
| Coating | |
| HPMC (e.g. Methocel E5) | 2.400 |
| Polyethylene Glycol 6000 | 0.700 |
| Titanium dioxide | 1.043 |
| Talc | 0.857 |
| Total Film coated tablet | 255.000 |

L) Film coated tablet

| Constituents | mg/tablet |
|---|---|
| Core | |
| Flibanserin | 20.000 |
| Lactose monohydrate | 130.000 |
| Microcrystalline cellulose | 43.100 |
| Hydroxypropyl Cellulose (e.g. Klucel LF) | 1.900 |
| Sodium Starch Glycolate | 4.000 |
| Magnesium stearate | 1.000 |
| Coating | |
| HPMC (e.g. Methocel E5) | 2.400 |
| Polyethylene Glycol 6000 | 0.700 |
| Titanium dioxide | 1.043 |
| Talc | 0.857 |
| Total Film coated tablet | 205.000 |

I claim:

1. A method for the treatment of a drug-induced sexual dysfunction comprising the administration of a therapeutically effective amount of flibanserin, or a pharmacologically acceptable acid addition salt thereof, or a hydrate or a solvate thereof.

2. The method for the treatment of a drug-induced sexual dysfunction according to claim 1, wherein the drug-induced sexual dysfunction is selected from the group consisting of drug-induced sexual desire disorders, drug-induced sexual arousal disorders, drug-induced orgasmic disorders, drug-induced sexual pain disorders and combinations thereof.

3. The method according to claim 1, wherein the drug-induced sexual dysfunction is a drug-induced sexual desire disorder.

4. The method according to claim 1, wherein the drug-induced sexual dysfunction is a drug-induced sexual arousal disorder.

5. The method according to claim 1, wherein the drug-induced sexual dysfunction is a drug-induced orgasmic disorder.

6. The method according to claim 1, wherein the drug-induced sexual dysfunction is a drug-induced sexual pain disorder.

7. The method according to claim 1, wherein the sexual dysfunction has been induced by a compound selected from the group consisting of α-adrenergic receptor antagonists, β-adrenergic receptor antagonists, calcium channel antagonists, sodium channel antagonists, 5-HT- and/or norepinephrine (NE) -reuptake inhibitors, 5-HT2A antagonists, D2 antagonists, dopamine agonists, Estrogen agonists, progesterone agonists, GABA agonists, HIV protease inhibitors, LH-RH modulators, MAO inhibitors, thiazide diuretics, potassium channel agonists, N-Methyl-D-aspartate (NMDA) receptor antagonists, muscarinic antagonists, Na/K-ATPase inhibitors, H/K-ATPase inhibitors, Histamine H1 or H2 antagonists, androgen antagonists, aldosterone antagonists, calmodulin antagonists, cholinergic receptor blockers, D2 agonists, 3-hydroxy-3-methyl-glutaryl-coenzyme A reductase inhibitors, progestins, GNRH inhibitors, GNRH analogues, antiestrogens and antiandrogens.

8. The method according to claim 1, wherein the sexual dysfunction has been induced by a compound selected from the group consisting of Prazosin, Clozapine, Mirtazapine, Reboxetine, Clonidine, Guanabenz, Guanethidine, Guanfacine, Guanadrel, Methyldopa, Phenoxibenzamine, Labetalol, Atenolol, Metoprolol, Nadolol, Timolol, Pindolol, Propranolol, Hydralazine, Phenytoin, Nifedipine, Verapamil, Amiodarone, Carbamazepine, Disopyramide, Nefazodone, Amfebutamone, Citalopram, Clomipramine, Fluoxetine, Mianserine, Paroxetine, Sertraline, Trazodone, Amitriptyline, Protriptyline, Amoxapine, Desipramine, Dothiepin, Imipramine, Nortriptyline, Venlafaxine, Reserpine, Fluvoxamine, Ziprasidone, Olanzapine, Droperidol, Metoclopramide, Pimozide, Sulpiride, Quetiapine, Risperidone, Thioridazine, Levodopa, Amineptine, Chlorpromazine, Flupentixol, Fluphenazine, Haloperidol, Perphenazineamitripyline, Levonorgestrel, ethinyl estradiol, Desogestrel, Lynoestrenol, Mestranol, Tamoxifene, Clonazepam, Valproate, Flurazepam, Phenobarbital, Primidone, Indinavir, Nelfinavir, Ritonavir, Saquinavir, Isocarboxazid, Moclobemide, Phenelzine, Tranylcypromine, Bendroflumethiazide, Chlortalidone, Hydrochlorothiazide, Trichlormethiazide, Indapamide, Minoxidil, Amantadine, Atropine, Bethanechol chloride, Digitalis, Digoxin, Esomeprazole, Doxepin, Cimetidine, Cyproterone acetate, Spironolactone, Trifluoperazine, Benzatropine, Bromocriptine, Reserpine, Triamterene, Stanozolol, Gemfibrozil, Disulfiram Lovastatin, Rosuvastatin, Fluvastatin, lovastatin, Atorvastatin, Simvastatin, Norethindrone, Norgestimate, Norgestrel, Drospirenone, Medroxyprogesterone, Abarelix, Triptorelin, Leuprolide, Anastrozol, Exemestane, Toremifine, Letrozole, Fulvestrant, Bicalutamide, Flutamide, or combination thereof.

9. The method according to claim 1, wherein flibanserin is in the form of a pharmaceutically acceptable acid addition salt wherein the pharmaceutically acceptable acid addition salt is formed by an acid selected from group consisting of succinic acid, hydrobromic acid, acetic acid, fumaric acid, maleic acid, methanesulphonic acid, lactic acid, phosphoric acid, hydrochloric acid, sulphuric acid, tartaric acid, citric acid, and mixtures thereof.

10. The method according to claim 1, wherein flibanserin is in the form of flibanserin polymorph A.

11. A kit comprising
a) a first pharmaceutical composition comprising an active ingredient, which has the side effect of causing a sexual dysfunction, for the treatment of an underlying disease;
b) a second pharmaceutical composition comprising flibanserin, or a pharmacologically acceptable acid addition salt thereof, or a hydrate or solvate thereof, for the treatment of the a sexual dysfunction induced by the active ingredient of the first pharmaceutical composition; and
c) a container for both compositions.

12. A pharmaceutical composition comprising a) an active ingredient which causes a sexual dysfunction as a side effect and b) flibanserin, or a pharmacologically acceptable acid addition salt thereof, or a hydrate or solvate thereof.
